(19) United States
(12) Patent Application Publication
(10) Pub. No.: US 2012/0029071 A1
(43) Pub. Date: Feb. 2, 2012

(54) COMPOSITIONS AND METHODS FOR TREATING OR PREVENTING RADIATION INJURY

(75) Inventors: Shyam Biswal, Ellicott City, MD (US); Rajesh K. Thimmulappa, Baltimore, MD (US)

(73) Assignee: THE JOHNS HOPKINS UNIVERSITY, Baltimore, MD (US)

(21) Appl. No.: 13/130,526

(22) PCT Filed: Nov. 23, 2009
(86) PCT No.: PCT/US09/06244

§ 371 Date: Sep. 29, 2011

Related U.S. Application Data
(62) Division of application No. 61/116,919, filed on Nov. 21, 2008.

(51) Int. Cl. A61K 31/275; A61P 1/00; A61P 25/00; A61P 13/12; C12N 5/077; A61P 1/12; A61P 1/08; A61P 17/00; A61P 19/00; C12N 5/071; A61P 7/00; A61P 17/02

(52) U.S. Cl. 514/527; 435/375

(57) ABSTRACT

The invention provides compositions and methods featuring Nrf2 activators for treating or preventing radiation-associated tissue damage.

### CROSS-REFERENCE TO RELATED APPLICATION

This application claims the benefit of the following U.S. Provisional Application No. 61/116,919, filed Nov. 21, 2008, the entire contents of which are incorporated herein by reference.

### STATEMENT OF RIGHTS TO INVENTIONS MADE UNDER FEDERALLY SPONSORED RESEARCH

This work was supported by the following grants from the National Institutes of Health, Grant Nos: HL081205 and GM079239. The government has certain rights in the invention.

### BACKGROUND OF THE INVENTION

Radiation injury can occur from external irradiation, either when the entire body is irradiated or when only part of the body is irradiated. Radiation injury may occur in connection with radiotherapy, during an accidental exposure to radioactivity, or in connection with a nuclear attack. Accidental exposure or nuclear attack can also cause internal radiation exposure due to widespread radioactive particles released in the environment. Radiation exposure causes short term and/or long term disorders. Clinical components of the acute radiation syndrome include the hematopoietic, gastrointestinal, and cerebrovascular syndromes that occur within days to a few weeks following radiation exposure. Long term disorders, such as lung fibrosis, following radiation exposure are typically associated with tissue damage. At present, there is no effective way to prevent or treat radiation injuries. Thus, improved compositions and methods for the treatment of radiation-associated tissue damage are urgently required.

### SUMMARY OF THE INVENTION

As described below, the present invention features compositions and methods for treating or preventing radiation-associated tissue damage.

In one aspect, the invention generally provides methods for treating or preventing cell damage associated with radiation exposure, the method involving contacting a cell with an effective amount of a Nrf2 activator (e.g., sulforaphane, triterpenoid).

In another aspect, the invention generally provides methods of preventing or reducing cell death associated with radiation injury, the method involving contacting a cell at risk of cell death with a Nrf2 activator, thereby preventing or reducing cell death relative to an untreated control cell.

In another aspect, the invention generally provides methods of treating or preventing radiation injury in a subject at risk thereof, the method involving administering to the subject an effective amount of a Nrf2 activator.

In yet another aspect, the invention generally provides packaged pharmaceutical comprising a therapeutically effective amount of a Nrf2 activator labeled for use in preventing or treating radiation injury, and instructions for use.

In still another aspect, the invention generally provides a kit for the amelioration of treating or preventing radiation injury comprising a Nrf2 activator and written instructions for use of the kit.

In various embodiments of the above aspects or any other aspect of the invention delineated herein, the method prevents or ameliorates hematopoietic syndrome, gastrointestinal syndrome, or cerebrovascular syndrome, pulmonary effects, renal failure, and effects on soft tissues. In one embodiment of the above aspects, the method prevents or ameliorates a symptom of hematopoietic damage that is any one or more of hypoplasia or aplasia of the bone marrow, pancytopenia, predisposition to infection, bleeding, and poor wound healing. In other embodiments of any of the above aspects, the method prevents or ameliorates a symptom of gastrointestinal damage that is any one or more of loss of intestinal crypts, breakdown of the mucosal barrier, abdominal pain, diarrhea, and nausea and vomiting. In still other embodiments of any of the above aspects, the method treats or prevents cutaneous injury from radiation burns wherein the injury is any one or more of loss of epidermis, loss of dermis, loss of muscle and loss of bone. In still other embodiments, the method prevents lung fibrosis or esophageal damage associated with radiotherapy. In still other embodiments of any of the above aspects, the method prevents or ameliorates inflammation. In still other embodiments, the compound is a compound listed in Table 1A (e.g., sulforaphane, a triterpenoid, such as Triterpenoid-155, Triterpenoid-156, Triterpenoid-162, and Triterpenoid-225). In still other embodiments, the radiation injury is associated with a nuclear attack or radiotherapy. In still other embodiments, the method increases Nrf2 transcription or translation. In still other embodiments, the method prevents cell death of a cell that is any one or more of a pulmonary cell, endothelial cell, pulmonary endothelial cell, smooth muscle cell, epithelial cell, and alveolar cell. In still other embodiments, the Nrf2 activator is administered before, during, or after radiation injury. In still other embodiments, the Nrf2 activator is administered within 1-12 (e.g., 1, 2, 3, 6, 9, 12) hours of radiation exposure. In still other embodiments, the Nrf2 activator is administered prior to radiation exposure.

The invention provides compositions and methods for treating or preventing radiation-associated tissue damage. Compositions and articles defined by the invention were isolated or otherwise manufactured in connection with the examples provided below. Other features and advantages of the invention will be apparent from the detailed description, and from the claims.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “Nrf2 activator” is meant any agent that increases Nrf2 expression or biological activity. Exemplary Nrf2 activators include but are not limited to small compounds, polypeptides and fragments thereof, and polynucleotides (e.g., DNA, RNA, microRNAs, siRNAs) and fragments thereof.

By “Nrf2 polypeptide” is meant a protein or protein variant, or fragment thereof, that comprises an amino acid sequence substantially identical to at least a portion of GenBank Accession No. NP_{—}006164 (human nuclear factor (erythroid-derived 2)-like 2) and that has a Nrf2 biological activity (e.g., activation of target genes through binding to antioxidant response element (ARE), regulation of expression of antioxidants and xenobiotic metabolism genes).

By “Nrf2 biological activity” is meant binding to an antioxidant-response element (ARE), nuclear accumulation, or the transcriptional induction of target genes.

By “Nrf2 nucleic acid molecule” is meant a polynucleotide encoding an Nrf2 polypeptide or variant, or fragment thereof.

By “Nrf2 nucleic acid molecule” is meant a polynucleotide encoding an Nrf2 polypeptide or variant, or fragment thereof.

By “radiation injury” is meant cell or tissue damage associated with exposure to ionizing radiation.

By “agent” is meant any small molecule chemical compound, antibody, nucleic acid molecule, or polypeptide, or fragments thereof.

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.”

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

In this disclosure, “comprises,” “comprising,” “containing” and “having” and the like can have the meaning ascribed to them in U.S. Patent law and can mean “ includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

By “disease” is meant any condition or disorder that damages or interferes with the normal function of a cell, tissue, or organ. Examples of diseases include radiation injury, as characterized by any reduction in cell or tissue biological function, including a reduction in hematopoiesis, gastrointestinal morphology or function, immune system function, lung function, renal function, central nervous system function, oral function and skin function.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The invention provides a number of targets that are useful for the development of highly specific drugs to treat or a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

As used herein, “obtaining” as in “obtaining an agent” includes synthesizing, purchasing, or otherwise acquiring the agent.

By “radiation injury” is meant any cell, tissue, or organ damage associated with radiation exposure. Examples of radiation injury include, but are not limited to, cerebrospinal injury, lung fibrosis, pneumonitis, hematopoietic injury, gastrointestinal injury, skin injuries and sepsis

By “reduces” is meant a negative alteration of at least 10%, 25%, 50%, 75%, or 100%.

By “reference” is meant a standard or control condition.

By “subject” is meant a mammal, including, but not limited to, a human or non-human mammal, such as a bovine, equine, canine, ovine, or feline.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

### BRIEF DESCRIPTION OF THE DRAWINGS

FIG. 1 is a graph showing mortality in Nrf2−/− mice and Nrf2+/+ after TBI. Mice (n=10/gp) mice were exposed to 9 Gy of TBI and survival was monitored for 21 days.

FIG. 2 includes three graphs, which provide proof of concept studies with Nrf2 activator, showing quantitation of TBI induced Mortality in Nrf2−/− mice and Nrf2+/+ mice after CDDO-me (small molecule Nrf2 activator) or vehicle treatment. One hour and 24 hours after TBI mice (n=10/gp) mice were treated with CDDO-Me. Survival was monitored for 30 days.

FIGS. 3A-3D show TBI induced GI injury in Nrf2+/+ and Nrf2−/− mice after CDDO-me or vehicle treatment. One hour after TBI (24 hours) mice (n=10/gp) mice were treated with CDDO-Me. GI injury was assessed by mucosal damage by histopathalogical analysis (FIGS. 3A and 3B) and crypt cell proliferation by BrdU immunohistochemistry staining (FIGS. 3C and 3D). FIGS. 3A and 3B are micrographs showing CDDO-Me treatment significantly mitigated reduction in gastrointestinal villi while 3C and 3D showing CDDO-Me significantly mitigated loss of crypt cells as indicated by increase proliferation of crypt cells.

FIG. 4 is a graph quantitating TBI induced HP injury in mice after CDDO-me or vehicle treatment as assessed by bone marrow clonogenic assay.

FIG. 5 is a table that quantifies Nrf2 regulated antioxidant defenses in multiple tissues of mice treated with CDDO-Me post TBI.

### DETAILED DESCRIPTION OF THE INVENTION

The invention features compositions and methods that are useful for the treatment of radiation injury.

The invention is based, at least in part, on the discovery that compounds that activate Nrf2 protect against cell and tissue damage associated with radiation exposure, and reduce mortality in response to such injury.

### Radiation Injury

Clinical components of acute radiation syndrome include hematopoietic, gastrointestinal, and cerebrovascular syndromes that occur within days or weeks of exposure. The hematopoietic syndrome, which is characterized by hypoplasia or aplasia of the bone marrow, occurs in connection with significant partial-body or whole-body radiation exposures. These hematopoietic changes result in pancytopenia, predisposition to infection, bleeding, and poor wound healing. Any one of these effects of radiation on hematopoiesis may be fatal. Gastrointestinal syndrome is characterized by abdominal pain, diarrhea, and nausea and vomiting and predispose patients to infection. Radiation induces loss of intestinal crypts and breakdown of the mucosal barrier. Cutaneous injury from thermal or radiation burns is characterized by loss of epidermis and dermis. Injuries to the skin may cover small areas but extend deep into the soft tissue, even reaching underlying muscle and bone.

### Mechanism of Radiation Injury

ROS and electrophiles generated by irradiation are key players in causing acute and chronic pathological injury. ROS induce oxidative damage to biomolecules and causes apoptosis of hematopoietic cells, endothelial cells and epithelial cells. Depletion of hematopoietic cells in a subject results in an impaired immune response and predisposes the subject to secondary infections. The increased death of endothelial cells and epithelial cells results in a loss of mucosal barrier and tissue injury. Loss of intestinal or lung mucosal barrier leads to translocation of bacteria into systemic circulation and causes systemic inflammation and sepsis. Tissue injury causes local inflammation leading to tissue remodeling and fibrosis. In sum, irradiation increases oxidative stress, apoptosis, and inflammation leading to multi-organ injury, which is often lethal. Therapies directed towards blocking ROS induced deleterious effects mitigates and treats radiation injury.

Nuclear Factor E2p45-Related Factor (Nrf2)

Nuclear factor erythroid-2 related factor 2 (NRF2), a cap-and-collar basic leucine zipper transcription factor, regulates a transcriptional program that maintains cellular redox homeostasis and protects cells from oxidative insult, including from chemotherapeutic agents (Rangasamy T, et al., J Clin Invest 114, 1248 (2004); Thimmulappa R K, et al. Cancer Res 62, 5196 (2002); So H S, et al. Cell Death Differ (2006)). NRF2 activates transcription of its target genes through binding specifically to the antioxidant-response element (ARE) found in those gene promoters.

Nrf2 is a redox sensitive transcription factor that regulates a battery of cellular antioxidant defenses that protect organisms from reactive oxygen species, inflammatory mediators and proapoptotic factors. Radiation injury is predominantly mediated by oxidative stress and inflammation. Radiation induces excess generation of reactive oxygen species and electrophiles that causes massive cell loss (e.g., loss of bone marrow stem cells, immune cells, epithelial cells and endothelial cells) in radiosensitive tissues (e.g., gut, liver, lung, brain, and kidney) due to apoptosis.

The present invention identifies Nrf2 as a novel drug target that can induce most powerful and efficient cellular antioxidant defense. Activation of Nrf2 prior to radiation exposure will help in preventing radiation injuries while postradiation activiation of Nrf2 mitigates and treats radiation injuries. The Nrf2 pathway can be manipulated to generate engineered stem cells. Nrf2 activators include pharmacological drugs (natural or synthetic), nucleic acid molecules (siRNA, miRNA)) that activate Nrf2 for the treatment or prevention of radiation induced disorders.

The NRF2-regulated transcriptional program includes a broad spectrum of genes, including antioxidants [γ-glutamyl cysteine synthetase modifier subunit (GCLm), γ-glutamyl cysteine synthetase catalytic subunit (GCLc), heme oxygenase-1, superoxide dismutase, glutathione reductase (GSR), glutathione peroxidase, thioredoxin, thioredoxin reductase, peroxiredoxins (PRDX), cysteine/glutamate transporter (SLC7A11) (7, 8)], phase II detoxification enzymes [NADP(H) quinone oxidoreductase 1 (NQO1), GST, UDP-glucuronosyltransferase (Rangasamy T, et al. J Clin Invest 114: 1248 (2004); Thimmulappa R K, et al. Cancer Res 62: 5196 (2002)), and several ATP-dependent drug efflux pumps, including MRP1, MRP2 (Hayashi A, et al. Biochem Biophy Res Commun 310: 824 (2003)); Vollrath V, et al. Biochem J (2006)); Nguyen T, et al. Annu Rev Pharmacol Toxicol 43: 233 (2003)).

### Nrf2 Activating Agents

Given that, increased Nrf2 expression or activity is useful for the treatment or prevention of radiation injury, agents that activate Nrf2 are useful in the methods of the invention. Such agents are known in the art and are described herein. Exemplary Nrf2 activating compounds include the class of compounds known as tricyclic bis-enones (TBEs) that are structurally related to synthetic triterpenoids, including RTA401 and RTA 402. Compounds useful in the methods of the invention include those described in U.S. Patent Publication No. 2004/002463, as well as those listed in Table 1A (below).

TABLE 1A

| Nrf2 activator | Year | Reference |
|---|---|---|
| 1,2,3,4,6-Penta-O-Galloyl- | 2006 | Mol Pharmacol. 2006 May; 69(5): 1554-63. Epub 2006 Jan. 31. |
| Beta-D-Glucose | | |
| 1,2-Diphenol (Catechol) | 2000 | J Biol Chem, Vol. 275, Issue 15, 11291-11299, Apr. 14, 2000 |
| 1,2-Dithiole-3-Thione | 2002 | J Biol Chem. 2003 Jan. 10; 278(2): 703-11. Epub 2002 Oct. 4. |
| 1,4-Diphenols | 2000 | J Biol Chem, Vol. 275, Issue 15, 11291-11299, Apr. 14, 2000 |
| (P-Hydroquinone) | | |
| 1-[2-Cyano-3-,12- | 2005 | Cancer Res. 2005 Jun. 1; 65(11): 4789-98. |
| Dioxooleana-1,9(11)-Dien- | | |
| 28-Oyl]Imidazole (CDDO-Im) | | |
| 15-Deoxy-12,14-Pgj2 | 2000 | J Biol Chem, Vol. 275, Issue 15, 11291-11299, Apr. 14, 2000 |
| 1-Chloro-2,4-Dinitrobenzene | 2000 | J Biol Chem. 2000 May 26; 275(21): 16023-9. |
| 2,3,7,8-Tetrachlorodibenzo- | 2003 | Cancer Res. 2003 Sep. 1; 63(17): 5636-45. |
| P-Dioxin | | |
| 2-Cyano-3,12-Dioxooleana- | 2005 | Biochem Biophys Res Commun. 2005 Jun. 17; 331(4): 993-1000. |
| 1,9(11)-Dien-28-Oic Acid | | |
| (CDDO) | | |
| 2-Indol-3-Yl- | 2003 | Biochem Biophys Res Commun. 2003 Aug. 8; 307(4): 973-9. |
| Methylenequinuclidin-3-Ols | | |
| 3-Hydroxyanthranilic Acid | 2006 | Drug Metab Dispos. 2006 January; 34(1): 152-65. Epub 2005 Oct. 21. |
| 3-Methylcholanthrene | 2006 | Febs J. 2006 June; 273(11): 2345-56. |
| 4-Hydroxyestradiol | 2003 | Mol Cell Biol. 2003 October; 23(20): 7198-209. |
| 4-Hydroxynonenal | 2005 | J Immunol. 2005 Oct. 1; 175(7): 4408-15. |
| 6-Methylsulfinylhexyl | 2002 | J. Biol. Chem., Vol. 277, Issue 5, 3456-3463, Feb. 1, 2002 |
| Isothiocyanate | | |
| 7-Oh Cmrn | 2001 | Cancer Research 61, 3299-3307, Apr. 15, 2001 |
| 9-Cis-Retinoic Acid | 2004 | Proc Natl Acad Sci USA. 2004 Mar. 9; 101(10): 3381-6. |
| | | Epub 2004 Feb. 25. |
| Acetaminophen | 2001 | Toxicol Sci. 2001 January; 59(1): 169-77. |
| Acetylcarnitine | 2004 | J Nutr. 2004 December; 134(12 Suppl): 3499s-3506s |
| Acrolein | 2002 | Free Radical Biology & Medicine, Vol. 32, No. 7, Pp. |
| | | 650-662, 2002 |
| Allyl Isothiocyanate | 2005 | J Invest Dermatol. 2005 April; 124(4): 825-32. |
| Alpha-Lipoic Acid | 2005 | Chem Res Toxicol. 2005 August; 18(8): 1296-305. |
| Apomorphine | 2006 | Ann N Y Acad Sci. 2006 May; 1067: 420-4. |
| Arsenic | 1999 | J. Biol. Chem., Vol. 274, Issue 37, 26071-26078, Sep. 10, 1999 |
| AUR ((2,3,4,6-Tetra-O)- | 2001 | J. Biol. Chem., Vol. 276, Issue 36, 34074-34081, Sep. 7, 2001 |
| Acetyl-1-Thio-D- | | |
| Glucopyranosato- | | |
| S)(Triethylphosphine) | | |
| Gold(I) | | |
| Autg ((1-Thio-D- | 2001 | J. Biol. Chem., Vol. 276, Issue 36, 34074-34081, Sep. 7, 2001 |
| Glucopyranosato) Gold(I) | | |
| Autm (Sodium | 2001 | J. Biol. Chem., Vol. 276, Issue 36, 34074-34081, Sep. 7, 2001 |
| Aurothiomalate | | |
| Avicins | 2004 | J Biol Chem. 2004 Mar. 5; 279(10): 8919-29. Epub 2003 Dec. 19. |
| Bis(2- | 2006 | Cell Death Differ. 2006 Feb. 17 |
| Hydroxybenzylidene)Acetone | | |
| Bleomycin | 2004 | Cancer Res. 2004 May 15; 64(10): 3701-13. |
| B-Naphthoflavone | 1998 | Oncogene (1998) 17, 3145 ± 3156 |
| Broccoli Seeds | 2004 | Free Radic Biol Med. 2004 Nov. 15; 37(10): 1578-90. |
| Bucillamine | 2006 | Biomaterials. 2006 Jun. 24; |
| Butylated Hydroxyanisole | 1997 | Biochemical And Biophysical Research Communications |
| | | 236, 313-322 (1997) |
| Butylated Hydroxytoulene | 1999 | PNAS U Oct. 26, 1999 U Vol. 96 U No. 22 U 12731-12736 |
| Cadmuim Chloride | 2000 | J Biol Chem. 2000 May 26; 275(21): 16023-9. |
| Cafestol | 2001 | Cancer Research 61, 3299-3307, Apr. 15, 2001 |
| Carbon Monoxide | 2006 | Cancer Lett. 2006 Mar. 3; |
| Carnosol | 2004 | J Clin Invest. 2004 November; 114(9): 1248-59. |
| Catechol | 2000 | J Biol Chem. 2000 May 26; 275(21): 16023-9. |
| Chlorogenic Acid | 2005 | Cancer Res. 2005 Jun. 1; 65(11): 4789-98. |
| Cigarette Smoke | 2003 | Pharm Res. 2003 September; 20(9): 1351-6. |
| Cobalt (Cobalt Chloride) | 2001 | J Biol Chem. 2001 Jul. 20; 276(29): 27018-25. Epub 2001 May 16. |
| Copper | 2006 | Drug Metab Dispos. 2006 January; 34(1): 152-65. Epub 2005 Oct. 21. |
| Coumarin | 2001 | Cancer Research 61, 3299-3307, Apr. 15, 2001 |
| Curcumin | 2003 | J Biol Chem. 2003 Feb. 14; 278(7): 4536-41. Epub 2002 Nov. 22. |
| Deprenyl (Selegiline) | 2006 | Carcinogenesis. 2006 May; 27(5): 1008-17. Epub 2005 Nov. 23. |
| Dexamethasone 21-Mesylate | 2002 | Toxicol Lett. 2002 Jun. 7; 132(1): 27-36. |
| Diallyl Disulfide | 2004 | Free Radic Biol Med. 2004 Nov. 15; 37(10): 1578-90. |
| Diallyl Sulfide | 2004 | Febs Lett. 2004 Aug. 13; 572(1-3): 245-50. |
| Diallyl Trisulfide (DATS) | 2004 | Free Radic Biol Med. 2004 Nov. 15; 37(10): 1578-90. |
| Diesel Exhaust | 2001 | J. Biol. Chem., Vol. 276, Issue 36, 34074-34081, Sep. 7, 2001 |
| Diethylmaleate | 2000 | J Biol Chem. 2000 May 19; 275(20): 15370-6. |
| Epicatechin-3-Gallate | 2001 | Drug Metabolism Reviews Volume 33, Number 3-4/2001 |
| Epigallocatechin-3-Gallate | 2005 | Arterioscler Thromb Vasc Biol. 2005 October; 25(10): 2100-5. |
| | | Epub 2005 Aug. 25. |
| Eriodictyo | 2006 | Invest Ophthalmol Vis Sci. 2006 July; 47(7): 3164-77. |
| Ethoxyquin. | 2000 | Biochem Soc Trans. 2000 February; 28(2): 33-41. |
| Ferulic Acid (Trans-4- | 2006 | Carcinogenesis. 2006 May; 27(5): 1008-17. Epub 2005 Nov. 23. |
| Hydroxy-3-Methoxycinnamic | | |
| Acid, 99% Purity) | | |
| Fisetin | 2006 | Invest Ophthalmol Vis Sci. 2006 July; 47(7): 3164-77. |
| Flunarizine | 2006 | World J Gastroenterol. 2006 Jan. 14; 12(2): 214-21. |
| Gallic Acid (3,4,5- | 2006 | Carcinogenesis. 2006 May; 27(5): 1008-17. Epub 2005 Nov. 23. |
| Trihydroxybenzoic Acid, | | |
| Gentisic Acid | 2006 | Carcinogenesis. 2006 May; 27(5): 1008-17. Epub 2005 Nov. 23. |
| Glucose Oxidase | 2000 | J Biol Chem. 2000 May 26; 275(21): 16023-9. |
| Glycosides From | 2006 | Food Chem Toxicol. 2006 August; 44(8): 1299-307. Epub 2006 |
| Digitalis Purpurea | | Mar. 6. |
| Heme | 1999 | J. Biol. Chem., Vol. 274, Issue 37, 26071-26078, Sep. 10, 1999 |
| Hemin | 2001 | J Biol Chem. 2001 May 25; 276(21): 18399-406. Epub 2001 Mar. 1. |
| Hydrogen Peroxide | 2000 | J Biol Chem. 2000 May 26; 275(21): 16023-9. |
| Hyerpoxia | 2005 | Free Radic Biol Med. 2005 Feb. 1; 38(3): 325-43. |
| Indole-3-Carbinol | 2001 | Cancer Research 61, 3299-3307, Apr. 15, 2001 |
| Indomethacin | 2002 | Drug Metabolism Reviews Volume 33, Number 3-4/2002 |
| Insulin | 2006 | Pharmazie. 2006 April; 61(4): 356-8. |
| Iodoacetic Acid | 2000 | J Biol Chem. 2000 May 26; 275(21): 16023-9. |
| Kahweol Palmitate | 2001 | Cancer Research 61, 3299-3307, Apr. 15, 2001 |
| Laminar Flow | 2002 | Proc Natl Acad Sci USA. 2002 Sep. 3; 99(18): 11908-13. |
| | | Epub 2002 Aug. 22. |
| Lead | 2006 | Drug Metab Dispos. 2006 January; 34(1): 152-65. Epub 2005 Oct. 21. |
| Limettin (LMTN) | 2001 | Cancer Research 61, 3299-3307, Apr. 15, 2001 |
| Lipoic Acid. | 2004 | J Clin Invest. 2004 January; 113(1): 65-73. |
| Lipopolysacharide | 2005 | Pharm Res. 2005 November; 22(11): 1805-20. Epub 2005 Aug. 16. |
| Luteolin | 2006 | J Neurosci Res. 2006 Jun. 26 |
| Lycopene | 2005 | J Neurosci Res. 2005 Feb. 15; 79(4): 509-21. |
| Menadione | 2000 | J Biol Chem. 2000 May 26; 275(21): 16023-9. |
| Mercury | 2006 | Am J Respir Cell Mol Biol. 2006 February; 34(2): 174-81. |
| | | Epub 2005 Sep. 29. |
| Nickel (II) | 2006 | Biochem Biophys Res Commun. 2006 Jul. 14; 345(4): 1350-7. |
| | | Epub 2006 May 15. |
| Nitric Oxide-Donating | 2005 | Atherosclerosis. 2005 Oct. 20; |
| Aspirin | | |
| Oltipraz | 2001 | Proc Natl Acad Sci USA. 2001 Mar. 13; 98(6): 3410-5 |
| Oxidized Low-Density | 2004 | Circ Res. 2004 Mar. 19; 94(5): 609-16. Epub 2004 Jan. 29. |
| Lipoproteins | | |
| Paraquat | 2000 | J Biol Chem. 2000 May 26; 275(21): 16023-9. |
| Parthenolide | 2005 | J Biochem Mol Biol. 2005 Mar. 31; 38(2): 167-76. |
| P-Coumaric Acid (Trans-4- | 2006 | Carcinogenesis. 2006 May; 27(5): 1008-17. Epub 2005 Nov. 23. |
| Hydroxycinnamic Acid), | | |
| Pgj2 | 2000 | J Biol Chem, Vol. 275, Issue 15, 11291-11299, Apr. 14, 2000 |
| Phenethyl Isothiocyanate | 2003 | Biochim Biophys Acta. 2003 Oct. 1; 1629(1-3): 92-101. |
| Phorbol 12-Myristate 13- | 2000 | Proc Natl Acad Sci USA. 2000 Nov. 7; 97(23): 12475-80. |
| Acetate (PMA) | | |
| P-Hydroxybenzoic Acid | 2006 | Carcinogenesis. 2006 April; 27(4): 803-10. Epub 2005 Nov. 2. |
| Proteasome Inhibitor MG-132 | 2006 | Biochem Pharmacol. 2006 Jun. 24; |
| Proteasome Inhibitors | 2003 | J Biol Chem. 2003 Jan. 24; 278(4): 2361-9. Epub 2002 Nov. 14. |
| (Lactacystin Or MG-132) | | |
| Pyrrolidine Dithiocarbamate | 2003 | Circ Res. 2003 Mar. 7; 92(4): 386-93. Epub 2003 Feb. 6. |
| Quercetin | 2006 | Invest Ophthalmol Vis Sci. 2006 July; 47(7): 3164-77. |
| Quercetin 3-O-Beta-L- | 2006 | Biochem Biophys Res Commun. 2006 May 12; 343(3): 965-72. |
| Arabinopyranoside | | Epub 2006 Mar. 29. |
| Resveratrol | 2005 | J Biochem Mol Biol. 2005 Mar. 31; 38(2): 167-76. |
| Sodium Arsenite | 2000 | J Biol Chem. 2000 May 26; 275(21): 16023-9. |
| Spermidine | 2003 | Toxicol Sci. 2003 May; 73(1): 124-34. Epub 2003 Mar. 25. |
| Spermine | 2003 | Biochem Biophys Res Commun. 2003 Jun. 6; 305(3): 662-70. |
| Spermine Nonoate | 2003 | Biochem Biophys Res Commun. 2003 Jun. 6; 305(3): 662-70. |
| Sulforaphane | 2002 | Cancer Res. 2002 Sep. 15; 62(18): 5196-203. |
| Sulforaphane | 2002 | J. Biol. Chem., Vol. 277, Issue 5, 3456-3463, Feb. 1, 2003 |
| Tert-Butylhydroquinone | 1998 | Oncogene (1998) 17, 3145 ± 3156 |
| (T-BHQ) | | |
| TNF-Alpha | 2005 | J Biol Chem. 2005 Jul. 29; 280(30): 27888-95. Epub 2005 Jun. 8. |
| Trans-Stilbene Oxide | 2006 | Biochem Biophys Res Commun. 2006 Jan. 20; 339(3): 915-22. |
| | | Epub 2005 Nov. 28. |
| Triterpenoid-155 | 2005 | Proc Natl Acad Sci USA. 2005 Mar. 22; 102(12): 4584-9. |
| | | Epub 2005 Mar. 14. |
| Triterpenoid-156 | 2005 | Proc Natl Acad Sci USA. 2005 Mar. 22; 102(12): 4584-9. |
| | | Epub 2005 Mar. 14. |
| Triterpenoid-162 | 2005 | Proc Natl Acad Sci USA. 2005 Mar. 22; 102(12): 4584-9. |
| | | Epub 2005 Mar. 14. |
| Triterpenoid-225 | 2005 | Mol Cancer Ther. 2005 January; 4(1): 177-86. |
| Tunicamycin | 2004 | Biochem J. 2004 Jan. 1; 377(Pt 1): 205-13. |
| Ultraviolet A Irradiation | 2005 | Proc Natl Acad Sci USA. 2005 Mar. 22; 102(12): 4584-9. |
| | | Epub 2005 Mar. 14. |
| Wasabi Extract | 2001 | Toxicol Appl Pharmacol. 2001 Jun. 15; 173(3): 154-60. |
| Xanthohumol (XH) | 2005 | Biochem J. 2005 Oct. 15; 391(Pt 2): 399-408. |
| Zerumbone | 2004 | Faseb J. 2004 August; 18(11): 1258-60. Epub 2004 Jun. 18. |
| Zinc | 1999 | J. Biol. Chem., Vol. 274, Issue 37, 26071-26078, Sep. 10, 1999 |

Library Screened: Spectrum 2000 and Sigma Lopac 1280
List of Activators

| | |
|---|---|
| 1 | Patulin |
| 2 | Methosyvone |
| 3 | Dehydrovariabilin |
| 4 | Biochanin A |
| 5 | Pdodfilox |
| 6 | 8-2′-Dimethoxyflavone |
| 7 | 6,3′-Dimethoxyflavone |
| 8 | Pinosylvin |
| 9 | Gentian Violet |
| 10 | Gramicidin |
| 11 | Thimerosal |
| 12 | Cantharidin |
| 13 | Fenbendazole |
| 14 | Mebendazole |
| 15 | Triacetylresveratrol |
| 16 | Resveratrol |
| 17 | Tetrachloroisopthalonitrile |
| 18 | Simvastatin |
| 19 | Valdecoxib |
| 20 | beta-Peltatin |
| 21 | 4,6-Dimethoxy-5-methylsioflavone |
| 22 | Nocodazole |
| 23 | Pyrazinecarboxamide |
| 24 | (±)-thero-1-Phenyl-2-decanoylamino-3- |
| | morpholino-1-propanol hydrochloride |
| 25 | SU4132 |

### Pharmaceutical Compositions

As reported herein, increased Nrf2 expression or biological activity is useful for the treatment or prevention of radiation injury and radiation-related cellular damage. Accordingly, the invention provides therapeutic compositions that increase Nrf2 expression in a cell, tissue, or organ, such as skin, lung, esophagus, or a gastrointestinal tissue.

An agent that increases Nrf2 expression or biological activity (e.g., a Nrf2 activator) may be administered within a pharmaceutically-acceptable diluents, carrier, or excipient, in unit dosage form. Conventional pharmaceutical practice may be employed to provide suitable formulations or compositions to administer the compounds to patients suffering from a disease that is associated with radiation injury and radiation-related cellular damage. Administration may begin before, during or after radiation exposure. In one embodiment, a Nrf2 activating agent is administered before, during or after radiotherapy (e.g., radiotherapy for the treatment of neoplasia). In another embodiment, a Nrf2 activating agent is administered after a nuclear attack (e.g., within 1, 2, 3, 4, 5, 6, 8, 10, 12 or 24 hours of exposure.

Any appropriate route of administration may be employed, for example, administration may be by inhalation, or parenteral, intravenous, intraarterial, subcutaneous, intratumoral, intramuscular, intracranial, intraorbital, ophthalmic, intraventricular, intrahepatic, intracapsular, intrathecal, intracisternal, intraperitoneal, intranasal, aerosol, suppository, or oral administration. For example, therapeutic formulations may be in the form of liquid solutions or suspensions; for oral administration, formulations may be in the form of tablets or capsules; and for intranasal formulations, in the form of powders, nasal drops, or aerosols.

Methods well known in the art for making formulations are found, for example, in “Remington: The Science and Practice of Pharmacy” Ed. A. R. Gennaro, Lippincourt Williams & Wilkins, Philadelphia, Pa., 2000. Formulations for parenteral administration may, for example, contain excipients, sterile water, or saline, polyalkylene glycols such as polyethylene glycol, oils of vegetable origin, or hydrogenated napthalenes. Biocompatible, biodegradable lactide polymer, lactide/glycolide copolymer, or polyoxyethylene-polyoxypropylene copolymers may be used to control the release of the compounds. Other potentially useful parenteral delivery systems for Nrf2 activators include ethylene-vinyl acetate copolymer particles, osmotic pumps, implantable infusion systems, and liposomes. Formulations for inhalation may contain excipients, for example, lactose, or may be aqueous solutions containing, for example, polyoxyethylene-9-lauryl ether, glycocholate and deoxycholate, or may be oily solutions for administration in the form of nasal drops, or as a gel.

The formulations can be administered to human patients in therapeutically effective amounts (e.g., amounts which prevent, eliminate, or reduce a pathological condition) to provide therapy for radiation injury. The preferred dosage of a Nrf2 activator of the invention is likely to depend on such variables as the type and extent of the disorder, the overall health status of the particular patient, the formulation of the compound excipients, and its route of administration.

With respect to a subject having radiation injury and/or radiation-related cellular damage, an effective amount is sufficient to increase Nrf2 expression or biological activity, increase antioxidant activity or reduce oxidative stress, or protect a cell, tissue or organism from damage or death. Generally, doses of Nrf2 activators would be from about 0.01 mg/kg per day to about 1000 mg/kg per day. It is expected that doses ranging from about 50 to about 2000 mg/kg will be suitable. Lower doses will result from certain forms of administration, such as intravenous administration. In the event that a response in a subject is insufficient at the initial doses applied, higher doses (or effectively higher doses by a different, more localized delivery route) may be employed to the extent that patient tolerance permits. Multiple doses per day are contemplated to achieve appropriate systemic levels of the compositions of the present invention.

A variety of administration routes are available. The methods of the invention, generally speaking, may be practiced using any mode of administration that is medically acceptable, meaning any mode that produces effective levels of the active compounds without causing clinically unacceptable adverse effects. Other modes of administration include oral, rectal, topical, intraocular, buccal, intravaginal, intracisternal, intracerebroventricular, intratracheal, nasal, transdermal, within/on implants, e.g., fibers such as collagen, osmotic pumps, or grafts comprising appropriately transformed cells, etc., or parenteral routes.

The present invention provides methods of treating radiation injury and radiation-related cellular damage or symptoms thereof which comprise administering a therapeutically effective amount of a pharmaceutical composition comprising a compound of the formulae herein to a subject (e.g., a mammal such as a human). Thus, one embodiment is a method of treating a subject suffering from or susceptible to a radiation injury and radiation-related cellular damageor symptom thereof. The method includes the step of administering to the mammal a therapeutic amount of an amount of a compound herein sufficient to treat the disease or disorder or symptom thereof, under conditions such that the disease or disorder is treated.

The methods herein include administering to the subject (including a subject identified as in need of such treatment) an effective amount of a compound described herein (e.g., Nrf2 activator), or a composition described herein to produce such effect. Identifying a subject in need of such treatment can be in the judgment of a subject or a health care professional and can be subjective (e.g. opinion) or objective (e.g. measurable by a test or diagnostic method).

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

The therapeutic methods of the invention (which include prophylactic treatment) in general comprise administration of a therapeutically effective amount of the compounds herein, such as a compound of the formulae herein to a subject (e.g., animal, human) in need thereof, including a mammal, particularly a human. Such treatment will be suitably administered to subjects, particularly humans, suffering from, having, susceptible to, or at risk for a disease, disorder, or symptom thereof. Determination of those subjects “at risk” can be made by any objective or subjective determination by a diagnostic test or opinion of a subject or health care provider (e.g., genetic test, enzyme or protein marker, Marker (as defined herein), family history, and the like). The compounds herein may be also used in the treatment of any other disorders in which radiation injury and radiation-related cellular damage may be implicated.

In one embodiment, the invention provides a method of monitoring treatment progress. The method includes the step of determining a level of diagnostic marker (Marker) (e.g., any target delineated herein modulated by a compound herein, a protein or indicator thereof, etc.) or diagnostic measurement (e.g., screen, assay) in a subject suffering from or susceptible to a disorder or symptoms thereof associated with radiation injury and radiation-related cellular damage, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

### Kits

The invention provides kits for preventing or treating radiation injury and/or radiation-related cellular damage (e.g., lung fibrosis). In one embodiment, the kit comprises a sterile container that contains a Nrf2 activator; such containers can be boxes, ampoules, bottles, vials, tubes, bags, pouches, blister-packs, or other suitable container form known in the art. Such containers can be made of plastic, glass, laminated paper, metal foil, or other materials suitable for holding nucleic acids. The instructions will generally include information about the use of the Nrf2 activator in treating or preventing oxidative stress or cellular damage associated with radiation injury and radiation-related cellular damage. Preferably, the kit further comprises any one or more of the reagents described in the assays described herein. In other embodiments, the instructions include at least one of the following: description of the Nrf2 activator; methods for using the enclosed materials for the treatment or prevention of a radiation injury and radiation-related cellular damage; precautions; warnings; indications; clinical or research studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### Nrf2 is a Primary Regulator of Antioxidant Genes

Nrf2 regulates a network of cytoprotective genes including antioxidants in different organs (lung, intestine, liver, brain) in response to chemical activators or stressors. The antioxidant associated genes regulated by Nrf2 include direct antioxidants (SOD1, heme oxygenase-1 (Hmox1), NQO1) and genes associated with glutathione pathway (glutathione peroxidase (Gpx), glutathione reductase, glutamate cystiene ligase (catalytic and modifier subunit), thioredoxin pathway (thioredoxin reductase (Txnrd1), peroxiredoxin (Prdx)), as well as NADPH-regenerating enzymes (glucose 6-phosphate dehydrogenase (G6PD), phosphogluconate dehydrogenase (Pgd), and maleic enzyme 1 (Me1)) and xenobiotic detoxification enzymes (such as glutathione S-transferase (GST). In addition, Nrf2 regulates several other genes listed in Table 1 that function in a concerted fashion along with antioxidants to attenuate pathological damage caused by reactive oxygen species (ROS), reactive nitrogen species (RNS), and electrophiles.

TABLE 1
NRF2-regulated Gene Functions

| Functions | Target genes |
|---|---|
| Direct antioxidants | Heme oxygenase-1, |
| | Ferritin, NQO1, SOD1 |
| Increase the levels of GSH synthesis and | GCLM, GCLC, GCS, |
| regeneration | GSR |
| Stimulate NADPH synthesis | G6PD, malic enzyme |
| Encode enzymes that directly inactivate | GSTs, UGTs, |
| oxidants or electrophiles | |
| Increases detoxification of H₂O₂, | GPX2, peroxiredoxin |
| peroxynitrite and oxidative damage by | |
| products (4HNE, lipid hydroperoxides) | |
| Enhance the recognition and repair and | |
| removal of damaged DNA | |
| Chaperone activity; Enhance the recognition, | Heat shock proteins |
| repair, and removal of damaged proteins | (HSP 70), Proteosome |
| | members |
| Enhance toxin export via the multidrug | MRP1 |
| response transporters | |
| Inhibits cytokine mediated inflammation | Leukotriene B4 12- |
| | hydroxydehydrogenase |
| i) Enhances phagocytosis of bacteria | CD36, MARCO |
| | (scavenger receptors) |
| ii) maintenance of tissue homeostasis and | |
| resolution of inflammatory lesions by | |
| clearance of apoptotic cells | |
| Regulates redox dependent innate immune | Suppress NF-KB |
| as well as adaptive immune response | signaling |

### Example 2

### Nrf2 Protects Tissues from TBI Induced Mortality

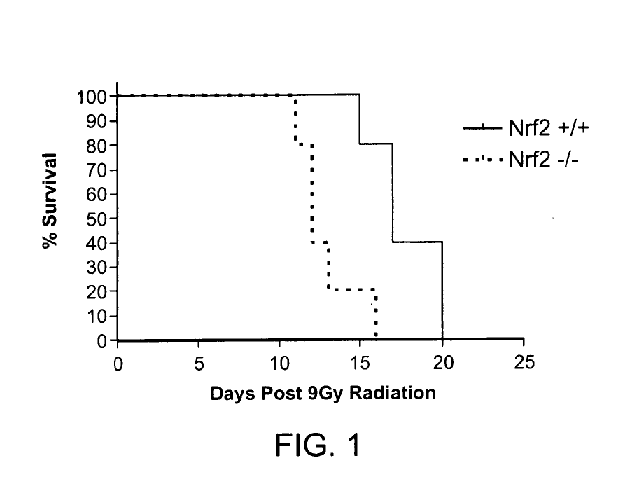

To determine the role of Nrf2 in survival following lethal total body irradiation (TBI), wild-type (Nrf2+/+) and Nrf2-deficient (Nrf2−/−) mice were exposed to 9 Gy. TBI induced early and greater mortality in Nrf2−/− mice when compared to Nrf2+/+ mice. These results indicate that Nrf2 improved survival following TBI (FIG. 1).

### Example 3

### Small Molecule Nrf2 Activator Significantly Mitigates TBI Induced Mortality in Nrf2+/+ Mice, but not in Nrf2−/− Mice

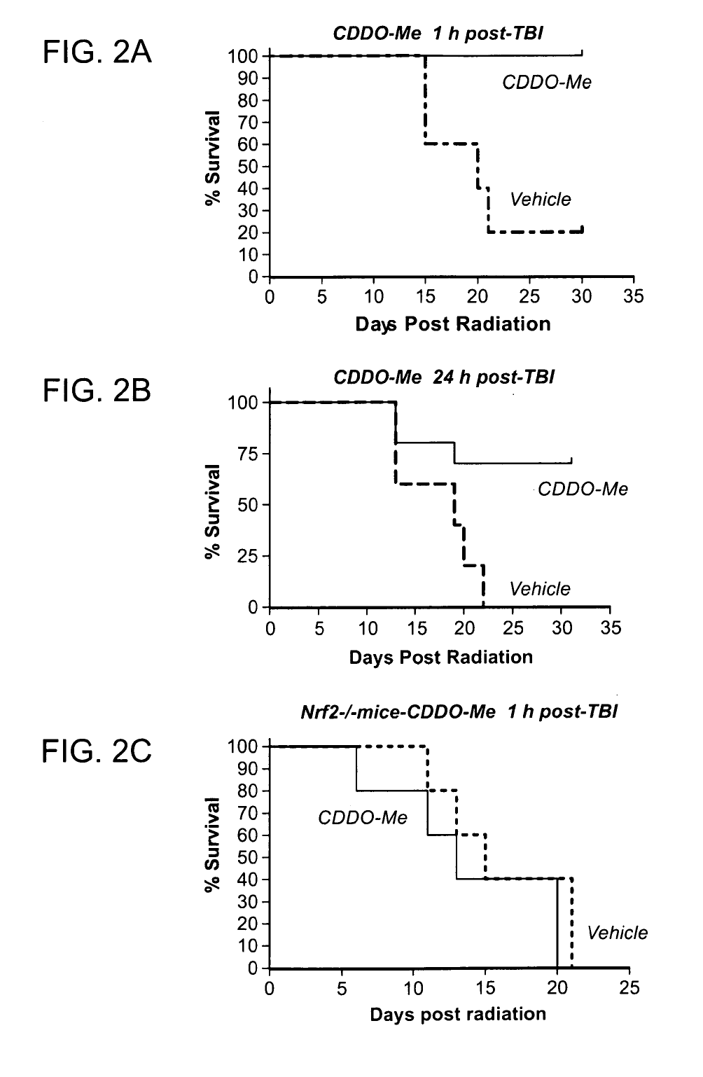

To determine if pharmacological activation of Nrf2 improves survival following TBI, we treated Nrf2+/+ and Nrf2−/− with the small molecule, CDDO-Me, a potent Nrf2 activator 1 hour and 24 hours after TBI. 100% mortality was observed in vehicle treated Nrf2+/+ mice. In contrast, CDDO-Me markedly inhibited TBI induced mortality in Nrf2+/+ mice. Mice treated with CDDO-Me 1 hour or 24 hours post-TBI showed 0% and 25% mortality, respectively. In contrast, CDDO-me treatment in Nrf2−/− mice 1 hour post-TBI failed to improve survival following TBI (FIG. 2).

### Example 4

### Nrf2 Activation by CDDO-me Mitigates TBI Induced Gastrointestinal (GI) Injury

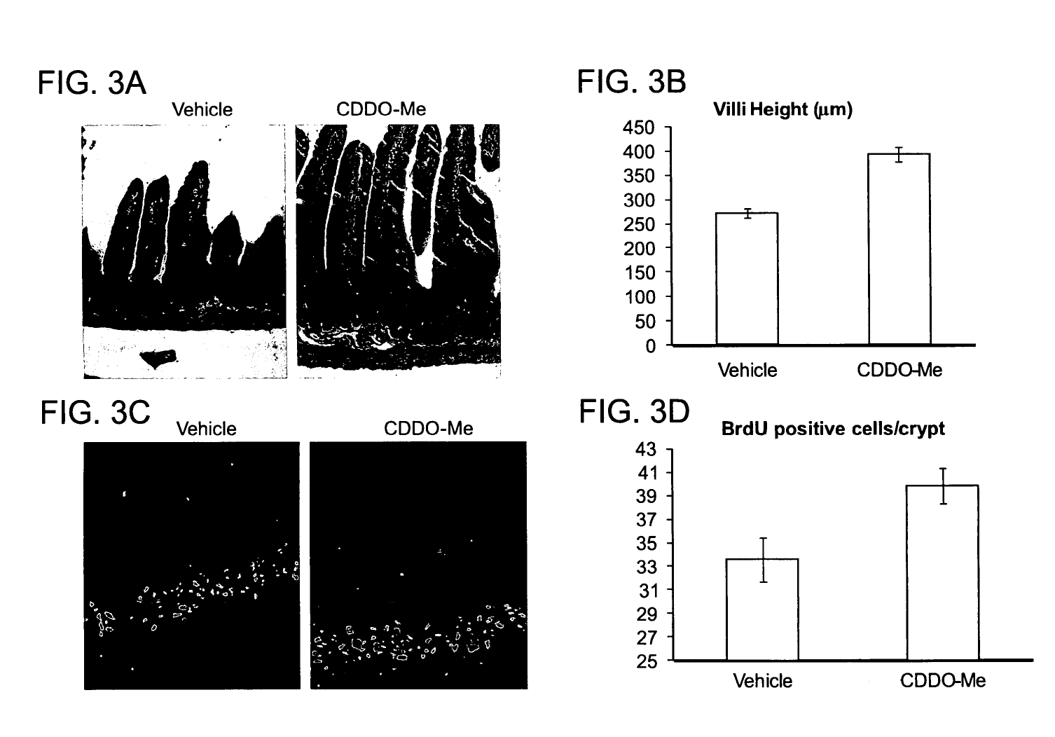

To analyze the GI injury, villi morphology and proliferation of crypt cells were quantified by immunohistochemistry and BrdU labeling on day 9 post TBI. CDDO-me treatment post 1 h TBI, markedly ameliorated mucosal injury as indicated greater length of villi when compared to vehicle treated group (FIGS. 3A & B). Furthermore, CDDO-Me treated group showed marked improvement in crypt cell proliferation as indicated by greater BrdU staining when compared to vehicle treated group (FIGS. 3C & D). In summary, activation of Nrf2 by CDDO-Me markedly protected from radiation induced intestinal damage.

### Example 5

### Activation of Nrf2 by Small Molecule Mitigates TBI Induced Hematopoietic (HP) Injury

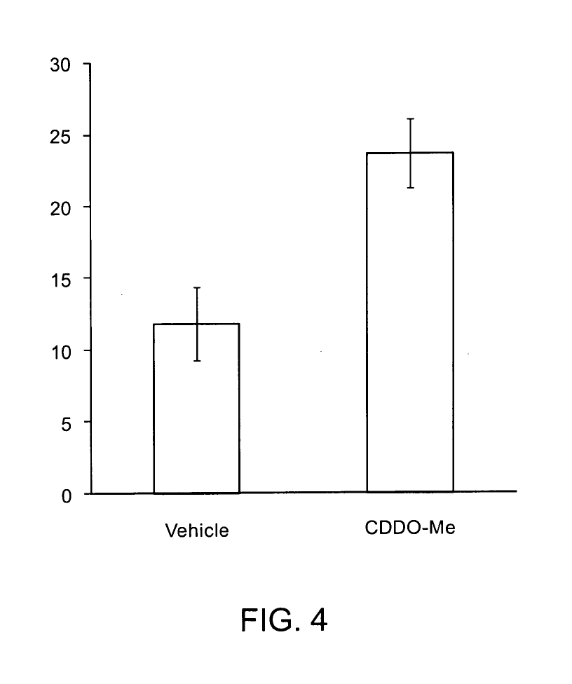

To assess the protective effect of CDDO-Me on hematopoietic injury, bone marrow was analyzed in a clonogenic assay. CDDO-Me treatment markedly protected hematopoietic stem cells and progenitors cell, as shown by the preservation of granulocyte/macrophage colony forming cells as shown in FIG. 4.

### Example 5

### Activation of Nrf2 by Small Molecule Improves Antioxidant Defenses in Multiple Organs Post TBI

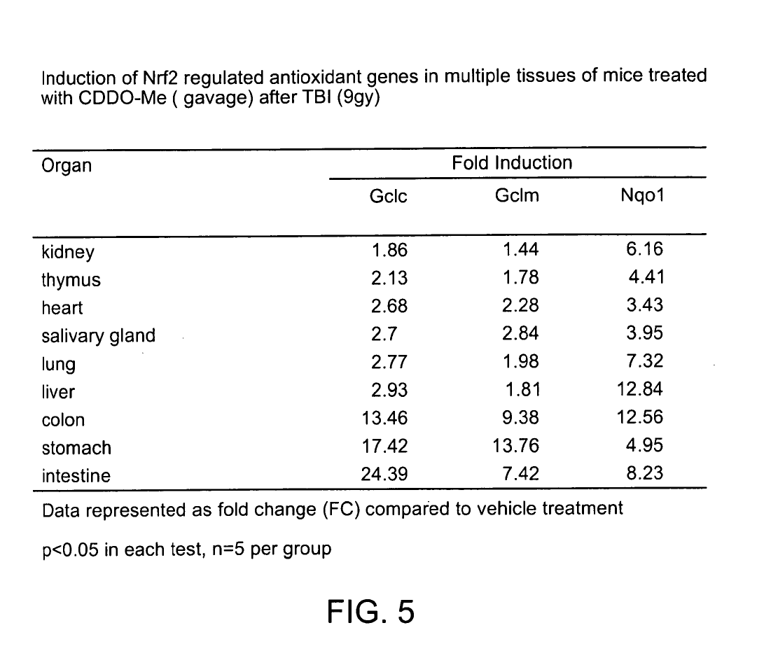

To analyze if activation of Nrf2 by CDDO-Me upregulated Nrf2 regulated antioxidant defenses post radiation, we measured expression of GCLC, GCLm and NQO1 genes in multiple tissues by quantative RT-PCR. CDDO-Me treatment markedly upregulated antioxidant genes in small intestine, colon, lung, liver, kidney, salivary gland and other organs (FIG. 5).

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

1. A method for treating or preventing cell damage associated with radiation exposure, the method comprising contacting a cell with an effective amount of a Nrf2 activator.

2. A method of preventing or reducing cell death associated with radiation injury, the method comprising contacting a cell at risk of cell death with a Nrf2 activator, thereby preventing or reducing cell death relative to an untreated control cell.

3. A method of treating or preventing radiation injury in a subject at risk thereof, the method comprising administering to the subject an effective amount of a Nrf2 activator.

4. The method of claim 1, wherein the method prevents or ameliorates hematopoietic syndrome, gastrointestinal syndrome, or cerebrovascular syndrome, pulmonary effects, renal failure, and effects on soft tissues,

5. The method of claim 4, wherein the method prevents or ameliorates a symptom of hematopoietic damage selected from the group consisting of hypoplasia or aplasia of the bone marrow, pancytopenia, predisposition to infection, bleeding, and poor wound healing.

6. The method of claim 4, wherein the method prevents or ameliorates a symptom of gastrointestinal damage selected from the group consisting of loss of intestinal crypts, breakdown of the mucosal barrier, abdominal pain, diarrhea, and nausea and vomiting.

7. The method of claim 1, wherein the method treats or prevents cutaneous injury from radiation burns wherein the injury is selected from the group consisting of loss of epidermis, loss of dermis, loss of muscle and loss of bone.

8. The method of claim 1, wherein the method prevents lung fibrosis or esophageal damage associated with radiotherapy.

9. The method of claim 4, wherein the method prevents or ameliorates inflammation.

10. The method of claim 1, wherein the compound is a compound listed in Table 1A.

11. The method of claim 10, wherein the compound is a triterpenoid.

12. The method off claim 10, wherein the compound is sulforaphane.

13. The method of claim 1, wherein the radiation injury is associated with a nuclear attack or radiotherapy.

14. The method of claim 1, wherein the method increases Nrf2 transcription or translation.

15. The method of claim 1, wherein the method prevents cell death of a cell selected from the group consisting of a pulmonary cell, endothelial cell, pulmonary endothelial cell, smooth muscle cell, epithelial cell, and alveolar cell.

16. The method of claim 1, wherein the Nrf2 activator is administered before, during, or after radiation injury.

17. The method of claim 1, wherein the Nrf2 activator is administered within 1-12 hours of radiation exposure.

18. The method of claim 1 wherein the Nrf2 activator is administered prior to radiation exposure.

19. A packaged pharmaceutical comprising a therapeutically effective amount of a Nrf2 activator labeled for use in preventing or treating radiation injury, and instructions for use.

20. A kit for the amelioration of treating or preventing radiation injury comprising a Nrf2 activator and written instructions for use of the kit.
